(19) United States
(12) Patent Application Publication
(10) Pub. No.: US 2016/0278977 A1
(43) Pub. Date: Sep. 29, 2016

(54) Cheek saver device

(71) Applicant: Francis J. Pansmith, Fort Lauderdale, FL (US)

(72) Inventor: Francis J. Pansmith, Fort Lauderdale, FL (US)

(21) Appl. No.: 14/545,090

(22) Filed: Mar. 24, 2015

(51) Int. Cl. A61F 7/12; A63B 71/08

(52) U.S. Cl. CPC A61F 7/12; A63B 71/085; A61F 2007/0017

(57) ABSTRACT

A device comprised of three sections, two outer cheek plates and a single inner guard molded into a single unit. The two cheek plates each house a reservoir chamber containing a coolant, and are molded to match the specific shape and contour of the inner cheek area. The inner guard provides insulation and contours to the teeth and gums.

This application refers to U.S. Provisional application No. 61/967,740 filing date Mar. 26, 2014

### BACKGROUND OF INVENTION

The current invention is a device to cool the inner mouth so as to affect the metabolism of the cheek and face, reducing over time the general loss of volume in the cheek area so as to produce a position cosmetic effect. For prior art, two areas that are of interest are devices to be placed in the mouth to affect change, and ice pack devices in general. Ice pack and devices for treating the inner mouth are known. Two U.S. Pat. No. 4,983,122 Mitnick and U.S. Pat. No. 6,217,606 Portonoy are general dental devices intended to treat trauma and surgical sites in the inner mouth area. Mitnick reveals a device somewhat unwieldy and has ice bags attached by Velcro or other means. Portnoy claims a specified device incorporating a substantially rectangular shape and relying on a fixing cotton, gauze, or tape for insulation. Cotton or gauze seem to be questionable options and the tape adhesive edge could attract and trap bacteria. Both devices lack the profile, thermal characteristics and general functionality of the current invention.

### REFERENCES CITED

U.S. Patent Documents

| | | |
|---|---|---|
| 3,780,537 | December 1973 | Spencer |
| 3,885,403 | May 1975 | Spencer |
| 4,196,904 | April 1980 | Borriello |
| 4,347,848 | September 1982 | Hubbard |
| 4,983,122 | January 1991 | Mitnick |
| 4,986,067 | January 1991 | Mitnick |
| 5,190,033 | March 1993 | Johnson |
| 5,624,257 | April 1997 | Farrel |
| 5,843,145 | December 1998 | Brink |
| 6,152,952 | November 2000 | Owens |
| 6,217,606 | April 2001 | Portnoy |
| 6,488,699 | December 2002 | Bypass |
| 6,610,084 | August 2003 | Torres |
| 7,416,516 | August 2008 | Mohindra |
| 8,881,734 | November 2014 | Wedemeyer |

### BRIEF DESCRIPTION OF THE DRAWINGS

FIG. 1 top view of device

FIG. 2 front view of device

FIG. 3 back view of device

FIG. 4 cheek plate inner guard cross section

FIG. 5 side profile enclosure/cheek plate

FIG. 6 back view cheek plate insert/cheek plate inner guard w/ profile gap

FIG. 7 cheek plate insert cross section

FIG. 8 top view mouth piece

FIG. 9 mouth piece/enclosure profile

FIG. 10 mouth piece sectional top view

### SPECIFICATION

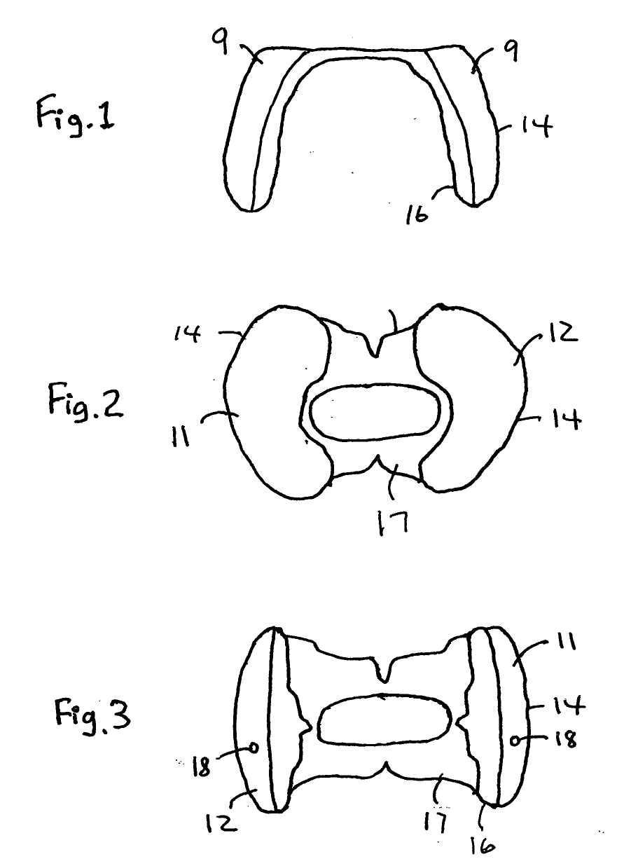

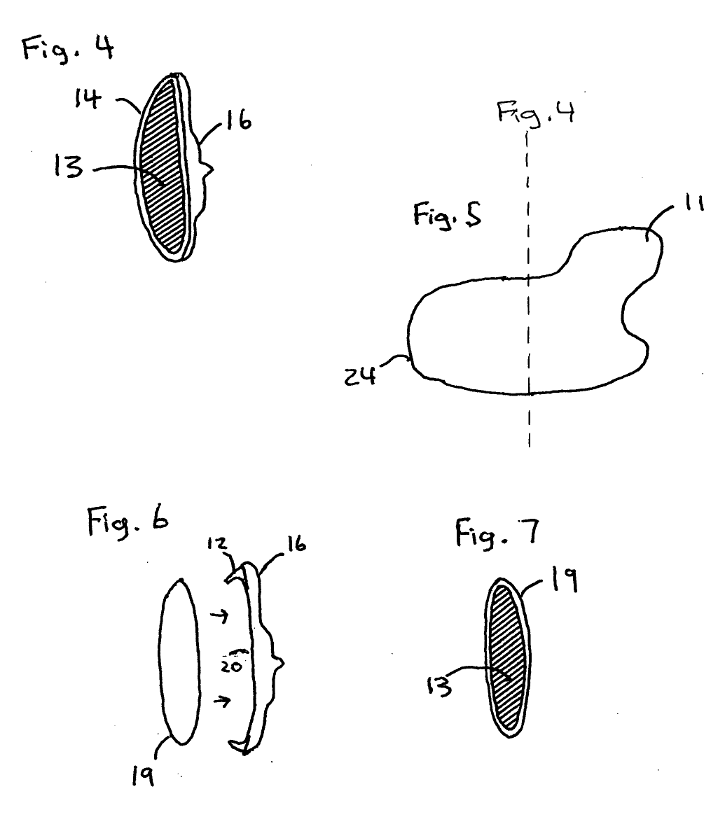

Referring to FIG. 1 of the drawings, a cheek saver 10 is shaped to fit in the vestibule of the mouth. The cheek saver having a right cheek plate 11 and a left cheek plate 12, comprised of a medical grade thermoplastic and a inner guard 16 comprised of a medical grade silicone rubber is molded whereas to form a single unit. FIGS. 2 and 3 further detail the general form. Detailing the design of a cheek plate 11 and 12, as shown in FIGS. 4 and 5, the cheek plates employ a unique profile designed to match the physiology of the inner cheek and mouth. The cross section view of FIG. 4 reveals an inner reservoir chamber 13, which is to be filled with a coolant. The coolant can be a commercially available non-toxic water, compound, or gel. FIG. 4 also reveals the exterior wall 14 of the cheek plate. The wall thickness of the exterior wall is significant in regard to the relative level of thermal transfer desired, which is determined by thicker wall equals less transfer, thinner wall equals more transfer. To be noted—although not limited to—the best mode concerning operational temperature radiated by the cheek saver in the range of 42 to 65 degrees Fahrenheit. Factoring into the equation is the thermal radiating properties of the particular thermoplastic being used, which can vary, and the particular coolant being employed, which also may vary. Having noted, it is revealed that the thickness of the exterior wall 14 is generally in the range of 0.5 mm to 2 mm. The inner guard 16 as shown in the drawings comprises the entire inner portion of the cheek saver, and is shaped to conform to the general form and physiology of the teeth and gums. Designed to provide an insulating barrier from the ice-cold contents of the reservoir chamber to the highly sensitive teeth and gums, the inner guard 16 as well as the cheek plates 11-12 may be custom fitted to any individual. It must be noted that the specified thickness of the exterior walls 14 may vary to the extent so as to provide for custom modeling to address facial imbalance. The cheek saver could also be manufactured from various materials such as vinyl, polyester, carbon, resin, ceramic, gallium, metal, or even glass so as the provide a more ornamental design item.

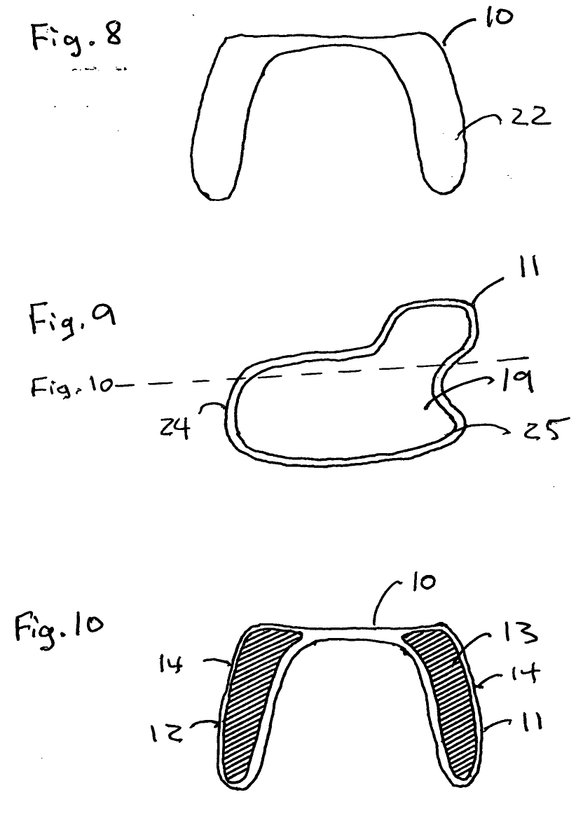

Another important design feature to be incorporated in the general embodiment of the invention is the option of the cheek plate insert 19. As shown in FIG. 6 the cheek plate insert, which now contains the coolant in its own interior reservoir chamber 13, shown in FIG. 7 is a separate entity which is designed to snap into place and further, be removable from the body of the cheek saver device. This has the benefits of convenience and interchangeability. The cheek plate 12 as shown in FIG. 6 embodies a profile gap 20, which is to receive the cheek plate insert 19. Another embodiment of the invention is the cheek saver device 10 to be molded in a singular form. FIGS. 8,9, and 10 detail the general form, which exactly matches the earlier embodiment, the difference is it being molded as a singular unit, with the exception of embodying the cheek plate insert option whereas it would then have three sections. Shown in FIG. 3 is a stop plug 18 or thermoplastic core plug to the interior of the cheek plate. It can be permanently sealed or a runner stop for filling and emptying the reservoir chamber.

### SUMMARY

The cheek saver device is comprised of three sections, two outer cheek plates and a single inner guard molded into a single unit. The two cheek plates each house a reservoir chamber containing a coolant, and are molded to match the specific shape and contour of the inner cheek area. The inner guard provides insulation and contours to the teeth and gums. The cheek saver device being previously placed in a freezer so as to chill the coolant, is intended to be placed in the vestibule of the mouth so as to provide a cooling effect to the inner cheek and face, therefore reducing the rate of fat burn off. The result is to limit over time, the general loss of volume and flesh reduction in the cheek area with repeated use of the device. The science of the process is fairly simple, whereas a sweat suit increases fat burn by increasing heat therefore raising metabolism, the opposite is realized when the current device is used to cool the cheek (which may become hot relative to other fatty sections of the body such as the belly area) therefore slowing the metabolism resulting in the stated objective. No other known device of product addresses this specific objective and method stated, other than plastic surgery or injectable substances. The cheek saver provides a safe natural wholesome remedy with no side effects.

I claim:

1. A apparatus for cooling the interior of the mouth, and having the effect of slowing the metabolism thereof, the apparatus comprising: a enclosure (9) wherein the improvement comprises the enclosure embodying a specific shape and profile (24) so as to match the physiology of the inner cheek and vestibule wherein the improvement comprises a more thorough and detailed affect so as to effect the facial area thereof; and the enclosure containing a reservoir chamber (13), the reservoir to house a coolant; and a inner guard (16) wherein the inner guard is shaped the fit the profile of the teeth and gums so as to facilitate proper placement and alignment whereas the apparatus is to be placed in the mouth.

2. The apparatus of claim 1 wherein the enclosure (9) comprises a cheek plate (11).

3. The apparatus of claim 1, further comprising: the cheek plates comprised of medical grade thermoplastic being plural in number including a right side (11) and a left side (12), having a exterior wall (14); and wherein the improvement comprises the exterior wall having a specified thickness whereby to regulate the degree of thermal transfer between the coolant and the area of the mouth being affected, furthermore the specified thickness of the exterior wall having a variance so as the provide various levels of thermal transfer to different specified areas of the mouth being affected.

4. The apparatus of claim 1 further comprising: the inner guard being comprised of medical grade silicon rubber being molded together with the cheek plates whereas to embody a single intergraded unit providing a more accurate and durable bond wherein the improvement comprises the bond reducing the opportunity for bacterial accumulation in the seams of the bond; and whereas the inner guard provides a full insulating barrier to the teeth and gums; and a inner guard front section (17) being flexible so as to facilitate in the insertion and removal process of the apparatus.

5. The apparatus of claim 1, further comprising: the apparatus being comprised of silicone, rubber compound, plastic, vinyl, polyester, carbon, resin, ceramic, gallium, glass, or metal in singular or in any combination thereof.

6. The apparatus of claim 1, further comprising: the coolant comprised of commercially available non-toxic water, compound, or gel.

7. The apparatus of claim 1, further comprising: the apparatus to be available in various sizes; and for the apparatus to be custom fitted and molded to match the precise measurements of an individual mouth.

8. The apparatus of claim 1, further comprising: a cheek plate insert (19) containing a reservoir chamber (13) the reservoir to house a coolant; and the cheek plate (12) to embody a profile gap (20) so as to receive the cheek plate insert whereas the cheek plate insert can snap into place within the cheek plate so as to provide for convenience and interchangeable applications.

9. A apparatus for cooling the interior of the mouth, the improvement comprising the cooling the effect of slowing the metabolism thereof, the apparatus comprising: a mouth piece (22) containing a reservoir chamber (13); and a coolant housed in the reservoir; a wherein the improvement comprises the mouth piece embodying a specific shape and profile (24) so as the match the physiology of the mouth; and wherein the improvement comprises the interior structure of the mouth piece and reservoir to have specified dimensions in the exterior wall (14) so as the determine various levels of thermal transfer to affect various specified areas of the mouth.

10. The apparatus of claim 9, further comprising: the apparatus being comprised of silicon, rubber compound, plastic, vinyl, polyester, carbon, resin, ceramic, gallium, glass or metal.

11. The apparatus of claim 9, further comprising: the coolant comprised of a commercially available non-toxic water, compound, or gel.

12. The apparatus of claim 9, further comprising: a cheek plate insert (19) containing a reservoir chamber (13) the reservoir to house a coolant; and the mouth piece (22) to employ a profile gap (25) so as to receive the cheek plate insert whereas the cheek plate insert can snap into place within the mouth piece so as to provide for convenience and interchangeable applications.
